(19) United States
(12) Patent Application Publication
(10) Pub. No.: US 2026/0120869 A1
(43) Pub. Date: Apr. 30, 2026

(54) METHOD AND SYSTEM FOR GENERATING DIAGNOSIS AND TREATMENT PATHWAYS BASED ON PATIENT CASE TWINS

(71) Applicant: Wipro Limited, Bangalore (IN)

(72) Inventors: Ashutosh BAJPAI, Haryana (IN); Noha EL-ZEHIRY, Princeton, NJ (US); Shivam SHARMA, Uttar Pradesh (IN); Manjunath Ramachandra IYER, Bangalore (IN); Addepalli Sai SRINIVAS, Hyderabad (IN)

(73) Assignee: Wipro Limited, Bangalore (IN)

(21) Appl. No.: 19/045,984

(22) Filed: Feb. 5, 2025

(30) Foreign Application Priority Data
Oct. 24, 2024 (IN) 202441080983

(51) Int. Cl. G16H 50/20; G16H 10/60; G16H 50/70

(52) U.S. Cl. CPC G16H 50/20; G16H 10/60; G16H 50/70

(57) ABSTRACT

A method for generating precision diagnosis and precision treatment based on patient case twins is disclosed. The method includes receiving query patient case from user device. The method may further include identifying first set of case twins corresponding to query patient case using retrieval model base on similarity analysis between set of query medical parameters and set of patient case medical parameters of each of plurality of patient cases stored in database. The method may further include identifying second set of case twins from first set of case twins using GenAI model. The method may further include determining precision diagnosis for query patient case based on query patient case and second set of case twins. The method may further include generating precision treatment pathway for query patient case based on query patient case, second set of case twins, and determined precision diagnosis using GenAI model.

The present application claims priority to Indian Patent Application number 202441080983, filed Oct. 24, 2024, which is incorporated herein by reference.

### DESCRIPTION

### Technical Field

This disclosure relates generally to medical assistance technologies, and more particularly to method and system for generating precision diagnosis and precision treatment pathways based on patient case twins.

### Background

The field of healthcare represents a significant and a complex area of focus for contemporary Generative Artificial Intelligence (GenAI) research due to various clinical challenges inherent in diagnosing medical conditions and determining effective, personalized treatments for the diagnosed medical conditions. In addition to the clinical challenges, there is a global shortage of medical professionals, particularly in rural areas, contributing to disparities in health equity. Precision in diagnosis and treatment is a critical component within a patient treatment lifecycle, especially given its implications and associated costs within the healthcare domain. Also, due to the burden of managing several cases, clinicians may struggle to stay updated with current literature and case histories.

In the present state of art, GenAI-based solutions (such as Large Language Models (LLMs), Large Multimodal Models (LMMs), and the like) aimed at addressing the above-mentioned challenges have helped to some extent. Efforts are underway to develop numerous clinical models built over foundation pre-trained LLMs, and LMMs. However, conventional Gen AI models may provide generic answers to clinical questions. The generic answers may provide some guidance to the clinicians however, these answers lack explainability, context, and personalization. Moreover, the conventional GenAI models may provide limited accuracy in diagnosis and personalized treatment recommendations, further limiting their mass adoption.

The present invention is directed to overcome one or more limitations stated above or any limitations associated with the known arts.

### SUMMARY

In one embodiment, a method for generating precision diagnosis and precision treatment pathways based on patient case twins is disclosed. In one example, the method may include receiving a query patient case from a user device. It should be noted that the query patient case may include a set of query medical parameters corresponding to a patient. The method may further include identifying a first set of case twins corresponding to the query patient case using a retrieval model based on a similarity analysis between the set of query medical parameters and a set of patient case medical parameters of each of a plurality of patient cases stored in a database. It should be noted that the first set of case twins may include a set of similar patient cases to the query patient case. It should be noted that the first set of similar patient cases is a subset of a plurality of patient cases stored in the database. Each of the plurality of patient cases may include a set of patient case medical parameters, patient case diagnosis, and patient case treatment pathway. The method may further include identifying a second set of case twins from the first set of similar patient cases using a Generative Artificial Intelligence (GenAI) model. The method may further include determining a precision diagnosis for the query patient case based on the query patient case and the second set of case twins using the GenAI model. The method may further include generating a precision treatment pathway for the query patient case based on the query patient case, the second set of case twins, and the determined precision diagnosis using the GenAI model.

In another embodiment, a system for generating precision diagnosis and precision treatment pathways based on patient case twins is disclosed. In one example, the system may include a processor and a computer-readable medium communicatively coupled to the processor. The computer-readable medium may store processor-executable instructions, which, on execution, may cause the processor to receive a query patient case from a user device. It should be noted that the query patient case may include a set of query medical parameters corresponding to a patient. The processor-executable instructions, on execution, may further cause the processor to identify a first set of case twins corresponding to the query patient case using a retrieval model based on a similarity analysis between the set of query medical parameters and a set of patient case medical parameters of each of a plurality of patient cases stored in a database. It should be noted that the first set of case twins may include a set of similar patient cases to the query patient case. It should be noted that the first set of similar patient cases is a subset of a plurality of patient cases stored in the database. Each of the plurality of patient cases may include a set of patient case medical parameters, patient case diagnosis, and patient case treatment pathway. The processor-executable instructions, on execution, may further cause the processor to identify a second set of case twins from the first set of case twins using a GenAI model. The processor-executable instructions, on execution, may further cause the processor to determine a precision diagnosis for the query patient case based on the query patient case and the second set of case twins using the GenAI model. The processor-executable instructions, on execution, may further cause the processor to generate a precision treatment pathway for the query patient case based on the query patient case, the second set of case twins, and the determined precision diagnosis using the GenAI model.

In yet another embodiment, a non-transitory computer-readable medium storing computer-executable instruction for generating precision diagnosis and precision treatment pathways based on patient case twins is disclosed. In one example, the stored instructions, when executed by a processor, may cause the processor to perform operations including receiving a query patient case from a user device. It should be noted that the query patient case may include a set of query medical parameters corresponding to a patient. The operations may further include identifying a first set of case twins corresponding to the query patient case using a retrieval model based on a similarity analysis between the set of query medical parameters and a set of patient case medical parameters of each of a plurality of patient cases stored in a database. It should be noted that the first set of case twins may include a set of similar patient cases to the query patient case. It should be noted that the first set of similar patient cases is a subset of a plurality of patient cases stored in the database. Each of the plurality of patient cases may include a set of patient case medical parameters, patient case diagnosis, and patient case treatment pathway. The operations may further include identifying a second set of case twins from the first set of case twins using a GenAI model. The operations may further include determining a precision diagnosis for the query patient case based on the query patient case and the second set of case twins using the GenAI model. The operations may further include generating a precision treatment pathway for the query patient case based on the query patient case, the second set of case twins, and the determined precision diagnosis using the GenAI model.

It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this disclosure, illustrate exemplary embodiments and, together with the description, serve to explain the disclosed principles.

FIG. 1 is a block diagram of an exemplary system for generating precision diagnosis and precision treatment pathways based on patient case twins, in accordance with some embodiments of the present disclosure.

FIG. 2 is a functional block diagram of a system for generating precision diagnosis and precision treatment pathways based on patient case twins, in accordance with some embodiments of the present disclosure.

FIGS. 3A and 3B are a flow diagram of an exemplary process for generating precision diagnosis and precision treatment pathways based on patient case twins, in accordance with some embodiments of the present disclosure.

FIG. 4 is a flow diagram of a detailed exemplary control logic for generating precision diagnosis and precision treatment pathways based on patient case twins, in accordance with an embodiment of the present disclosure.

FIG. 5 is a flow diagram of an exemplary process for fine-tuning a retrieval model, in accordance with some embodiments of the present disclosure.

FIG. 6 is a flow diagram of a detailed exemplary process for fine-tuning a retrieval model, in accordance with an embodiment of the present disclosure.

FIG. 7 is a flow diagram of a detailed exemplary process for training data construction based on predefined weightage variants, in accordance with an embodiment of the present disclosure.

FIG. 8 is a block diagram of an exemplary computer system for implementing embodiments consistent with the present disclosure.

### DETAILED DESCRIPTION

Exemplary embodiments are described with reference to the accompanying drawings. Wherever convenient, the same reference numbers are used throughout the drawings to refer to the same or like parts. While examples and features of disclosed principles are described herein, modifications, adaptations, and other implementations are possible without departing from the spirit and scope of the disclosed embodiments. It is intended that the following detailed description be considered as exemplary only, with the true scope and spirit being indicated by the following claims.

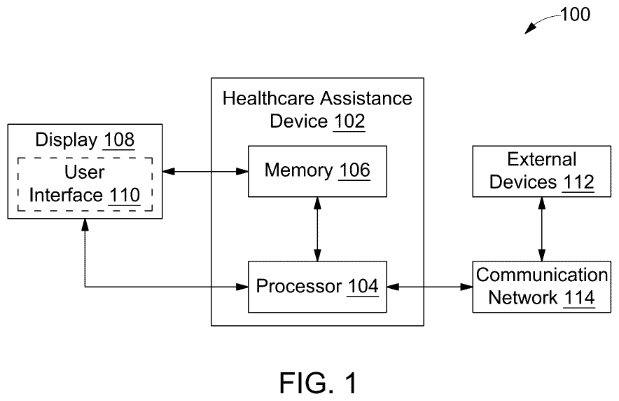

Referring now to FIG. 1, an exemplary system 100 for generating precision diagnosis and precision treatment pathways based on patient case twins is illustrated, in accordance with some embodiments of the present disclosure. The system 100 may include a healthcare assistance device 102. The healthcare assistance device 102 may be, for example, but may not be limited to, server, desktop, laptop, notebook, netbook, tablet, smartphone, mobile phone, or any other computing device, in accordance with some embodiments of the present disclosure. The healthcare assistance device 102 may determine a precision diagnosis and a precision treatment pathway for a query patient case using a Generative Artificial Intelligence (GenAI) model based on an identified patient case twin.

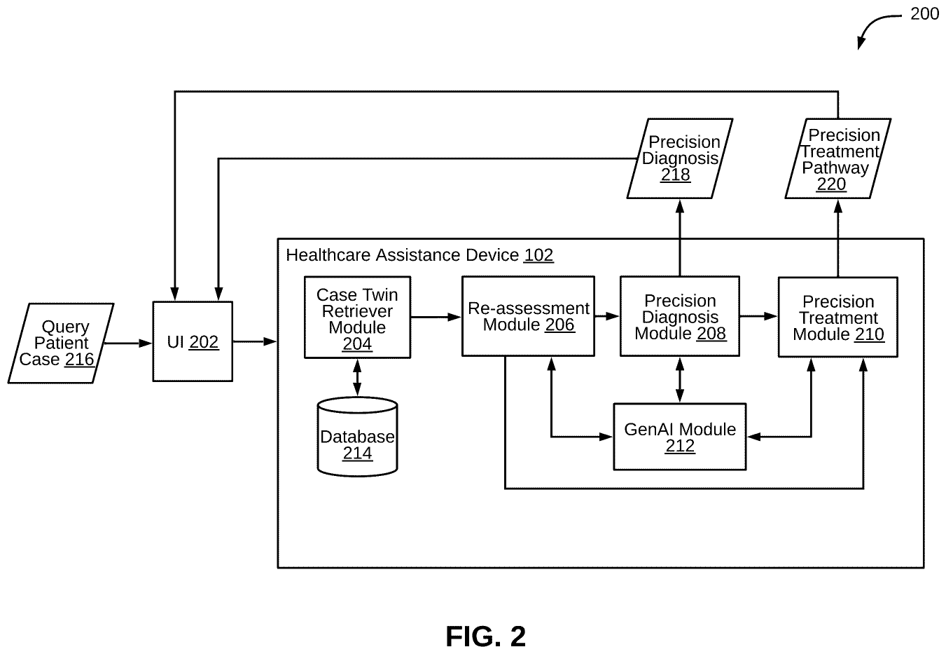

As will be described in greater detail in conjunction with FIGS. 2-8, the healthcare assistance device 102 may receive a query patient case from a user device. It should be noted that the query patient case may include a set of query medical parameters corresponding to a patient. The healthcare assistance device 102 may further identify a first set of case twins corresponding to the query patient case using a retrieval model based on a similarity analysis between the set of query medical parameters and a set of patient case medical parameters of each of a plurality of patient cases stored in a database. It should be noted that the first set of case twins may include a set of similar patient cases to the query patient case. It should also be noted that the first set of similar patient cases is a subset of a plurality of patient cases stored in the database. Each of the plurality of patient cases may include a set of patient case medical parameters, patient case diagnosis, and patient case treatment pathway. The healthcare assistance device 102 may further identify a second set of case twins from the first set of case twins using a GenAI model. The healthcare assistance device 102 may further determine a precision diagnosis for the query patient case based on the query patient case and the second set of case twins using the GenAI model. The healthcare assistance device 102 may further generate a precision treatment pathway for the query patient case based on the query patient case, the second set of case twins, and the determined precision diagnosis using the GenAI model.

In some embodiments, the healthcare assistance device 102 may include one or more processors 104 and a memory 106. Further, the memory 106 may store instructions that, when executed by the one or more processors 104, may cause the one or more processors 104 to generate precision diagnosis and precision treatment pathways based on patient case twins, in accordance with aspects of the present disclosure. The memory 106 may also store various data (for example, a first set of case twins, a second set of case twins, a plurality of patient cases, a plurality of query medical parameter embeddings, a plurality of patient case medical parameter embeddings, a set of patient case medical parameters, and the like) that may be captured, processed, and/or required by the system 100. The memory 106 may be a non-volatile memory (e.g., flash memory, Read Only Memory (ROM), Programmable ROM (PROM), Erasable PROM (EPROM), Electrically EPROM (EEPROM) memory, etc.) or a volatile memory (e.g., Dynamic Random Access Memory (DRAM), Static Random-Access memory (SRAM), etc.).

The system 100 may further include a display 108. The system 100 may interact with a user interface 110 accessible via the display 108. The system 100 may also include one or more external devices 112. In some embodiments, the healthcare assistance device 102 may interact with the one or more external devices 112 over a communication network 114 for sending or receiving various data. The communication network 114 may include, for example, but may not be limited to, a wireless fidelity (Wi-Fi) network, a light fidelity (Li-Fi) network, a local area network (LAN), a wide area network (WAN), a metropolitan area network (MAN), a satellite network, the internet, a fiber optic network, a coaxial cable network, an infrared (IR) network, a radio frequency (RF) network, and a combination thereof. The one or more external devices 112 may include, but may not be limited to, a remote server, a laptop, a netbook, a notebook, a smartphone, a mobile phone, a tablet, or any other computing device.

Referring now to FIG. 2, a functional block diagram of a system 200 for generating precision diagnosis and precision treatment pathways based on patient case twins is illustrated, in accordance with some embodiments of the present disclosure. FIG. 2 is explained in conjunction with FIG. 1. The system 200 may be analogous to the system 100. The system 200 may include the healthcare assistance device 102 and a user interface (UI) 202. The healthcare assistance device 102 may include, within the memory 106, a case twin retrieval module 204, a re-assessment module 206, a precision diagnosis module 208, a precision treatment module 210, a GenAI module 212, and a database 214.

In an embodiment, the UI 202 may be rendered on a display (such as the display 108 of the healthcare assistance device 102. In an alternative embodiment, the UI 202 may be rendered on a user device (for example, a laptop, a mobile phone, a notebook, a netbook, a smartphone, or any other computing device). In such an embodiment, the user device may be communicatively connected to the healthcare assistance device 102. The UI 202 may be a Graphical UI (GUI). The UI 202 may be, for example, but may not be limited to, a text-based user interface or a voice-based user interface with a speech-to-text conversion capability.

The UI 202 may be accessed by a user. The user may be, for example, but may not be limited to, a doctor, a clinician, a physician specialist, or a surgeon. The user may provide a query patient case 216 corresponding to a patient through the UI 202. The query patient case 216 may be received in a format of, for example, Portable Document Format (PDF), word document format (DOC or DOCX), Text file format (TXT), database records, and the like. In some embodiments, the query patient case 216 may also include multi-modal data (such as images, video frames, etc.). By way of an example, the multi-modal data corresponding to the patient may include, but may not be limited to, ultrasound images, Magnetic Resonance Imaging (MRI) images, X-ray images, Computed Tomography (CT) scans, and the like.

The query patient case 216 may include information (i.e., a set of query medical parameters) corresponding to the patient. By way of an example, the set of query medical parameters may include, but may not be limited to, demographic information, medical history, symptoms, physical examination information, and laboratory test results. Additionally, the set of query medical parameters may include may also include a patient identifier (ID). The patient ID may be in one of, a numeric value, an alpha-numeric value, or a roman number Further, the UI 202 may send the query patient case 216 to the case twin retrieval module 204, the re-assessment module 206, the precision diagnosis module 208, and the precision treatment module 210.

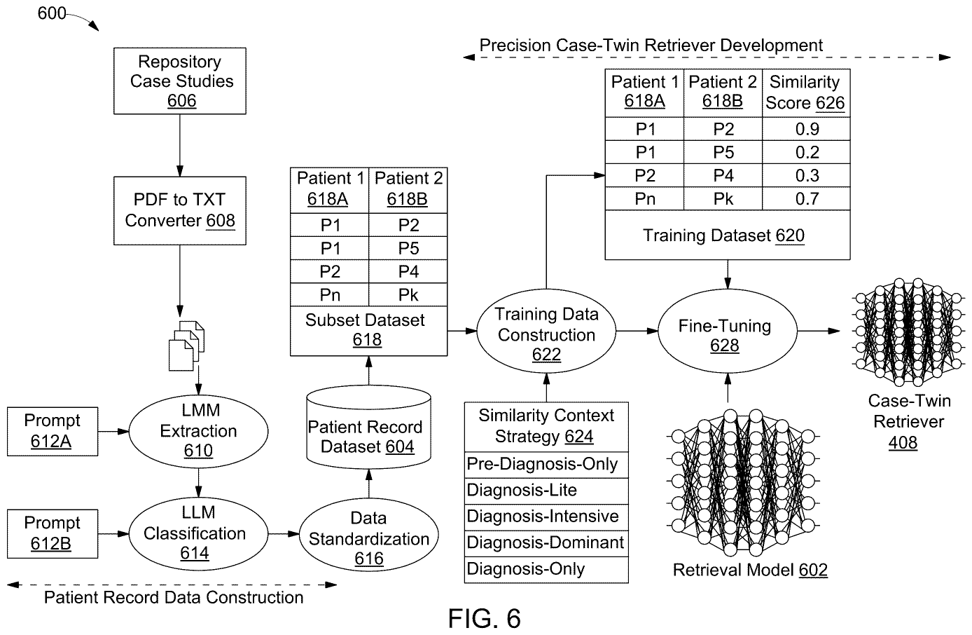

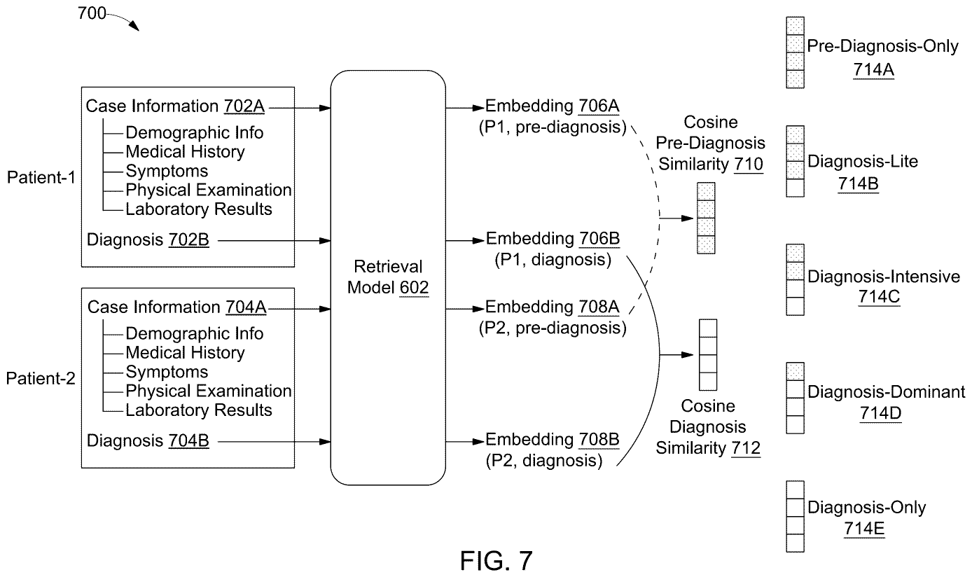

The case twin retrieval module 204 may receive the query patient case 216 from a user through the UI 202. Further, the case twin retrieval module 204 may identify a first set of case twins corresponding to the query patient case 216 using a pre-trained retrieval model based on a similarity analysis between the set of query medical parameters and a set of patient case medical parameters of each of a plurality of patient cases stored in the database 214. The retrieval model may be, for example, but may not be limited to, a Bidirectional Encoder Redirection Transformer (BERT), a clinicalBERT, a bioBERT, and a PubMedBERT. This is further explained in greater detail in conjunction with FIGS. 6-7.

It should be noted that the first set of case twins may include a set of similar patient cases to the query patient case. The first set of similar patient cases is a subset of a plurality of patient cases stored in an Electronic Health Record (EHR) or a Patient Record Database. The EHR or the Patient Record Database may be stored in the database 214.

To identify the first set of case twins, the case twin retrieval module 204, may create a plurality of query medical parameter embeddings from the set of query medical parameters using an embedding model. The embedding model may be, for example, but may not be limited to, Word2Vec, Glove, or BERT. Further, the case twin retrieval module 204 may retrieve, via the pre-trained retrieval model, a plurality of patient case medical parameters embeddings corresponding to the plurality of patient cases from the database 214. It should be noted that each of the plurality of patient cases may include a set of patient case medical parameters, patient case diagnosis, and patient case treatment pathway. The set of patient case medical parameters may correspond to the set of query medical parameters. Thus, for example, the set of patient case medical parameters may include, but may not be limited to, demographic information, medical history, symptoms, physical examination information, and laboratory test results. In an embodiment, the plurality of patient case medical parameter embeddings may be created by the embedding model and pre-stored in the database 214.

Further, the case twin retrieval module 204 may calculate a first similarity score between the query patient case 216 and each of the plurality of patient cases. The first similarity score may be calculated based on a similarity analysis between the plurality of query medical parameter embeddings and a corresponding plurality of patient case medical parameter embeddings of each of the plurality of patient cases. The similarity analysis may be based on a distance function, for example, but not limited to, cosine similarity, Euclidean distance, Jaccard similarity, Minkowski distance, and Manhattan distance.

Upon calculating the first similarity score, the case twin retrieval module 204 may identify the first set of case twins based on the first similarity score. The number (or range) for the first set of closest case twins may be pre-defined or configurable by the user. The case twin retrieval module 204 may select the predefined number of patient cases from the plurality of patient cases, each having the similarity score higher than each remaining patient case of the plurality of patient cases. Further, the first set of case twins (along with the set of patient case medical parameters, patient case diagnosis, and patient case treatment pathway) may be retrieved from the database 214. Further, the case twin retrieval module 204 may send the retrieved first set of case twins to the re-assessment module 206.

The re-assessment module 206 may identify a second set of case twins from the first set of case twins through the GenAI module 212, using a GenAI model. The GenAI model may be, for example, but may not be limited to, Large Language Model (LLM), or a Large Multimodal Model (LMM). To identify the second set of case twins, the re-assessment module 206 may provide a case identification prompt to the GenAI module 212. The case identification prompt may include the first set of case twins, the set of query medical parameters, and a set of instructions corresponding to the second set of case twins. The set of instructions (also referred to as “re-assessment prompt template”) may be pre-stored in the database 214. The set of instructions may include instructions to provide the first similarity score between the query patient case 216 and each of the case twins within the first set of case twins. Additionally, the set of instructions may include instructions to provide a sorted list (or an ordered list) of the first set of case twins where a first case twin in the sorted list represents the most similar patient case with the query patient case 216 and the last case in the sorted list represents the least similar patient case with the query patient case.

To create the case identification prompt, the re-assessment module 206 may add the first set of case twins and the query patient case 216 to the pre-stored set of instructions (i.e., the re-assessment prompt template). Once the case identification prompt is created, the re-assessment module 206 may send the case identification prompt to the GenAI module 212.

The GenAI module 212 may include the GenAI model. Alternatively, the GenAI module 212 may fetch the GenAI model from an external server. The GenAI module 212 may generate, via the GenAI model, the sorted list of the first set of case twins based on the case identification prompt. The sorted list may include a patient case identifier (ID) of each of the first set of patient case twins and a second similarity score between the query patient case 216 and each of the first set of patient cases. The patient case ID may be, for example, a numeric value, an alphabetic character, a roman numeral, or an alpha-numeric value, and the like. The second similarity score is calculated by the GenAI model. It should be noted that the first set of case twins in the sorted list is arranged based on the second similarity score. Upon generating the sorted list, the GenAI module 212 may send the sorted list to the re-assessment module 206.

Further, the re-assessment module 206 may compare the second similarity score of each of the first set of case twins with a predefined threshold similarity score. Further, the re-assessment module 206 may truncate the sorted list based on the comparison to obtain the second set of case twins. In other words, the sorted list may be truncated using the predefined threshold similarity score to select the most similar case twins from the first set of case twins. Further, the re-assessment module 206 may send the second set of case twins to the precision diagnosis module 208.

The precision diagnosis module 208 may determine a precision diagnosis 218 for the query patient case 216 based on the query patient case 216 and the second set of case twins through the GenAI module 212, using the GenAI model. To determine the precision diagnosis 218, the precision diagnosis module 208 may create a precision diagnosis determination prompt. The precision diagnosis determination prompt may include the set of query medical parameters, the second set of case twins, and a set of diagnosis instructions (also referred to as “precision diagnosis prompt template).

The set of diagnosis instructions may be pre-stored in the database 214. The set of diagnosis instructions may include instructions to provide the precision diagnosis 218 based on information from the query patient case 216 and the second set of case twins. In an embodiment, the set of diagnosis instructions may be provided in a question-answer format (i.e., labelled data) that may guide the GenAI model to provide the precision diagnosis 218 in the same format as the diagnosis of each of the second set of case twins. Further, the precision diagnosis module 208 may add the query patient case 216 and the second set of case twins to the set of diagnosis instructions to obtain the precision diagnosis determination prompt.

Further, the precision diagnosis module 208 may send the precision diagnosis determination prompt to the GenAI module 212. Further, the GenAI module 212 may generate a response to the precision diagnosis determination prompt using the GenAI model. The response may include the precision diagnosis 218 for the query patient case 216. Further, the GenAI module 212 may send the response (i.e., the precision diagnosis for the query patient case) to the precision diagnosis module 208. Upon receiving the response, the precision diagnosis module 208 may then present the precision diagnosis 218 on the UI 202. Further, the precision diagnosis module 208 may send the second set of case twins and the precision diagnosis 218 for the query patent case 216 to the precision treatment module 210.

The precision treatment module 210 may generate a precision treatment pathway 220 for the query patient case 216 based on the query patient case 216, the second set of case twins, and the determined precision diagnosis 218 through the GenAI module 212, using the GenAI model. To generate the precision treatment for the query patient case 216, the precision treatment module 210 may provide a precision treatment generation prompt to the GenAI module 212. The precision treatment generation prompt may include the set of query medical parameters, the second set of case twins, and a set of treatment instructions (also referred to as “precision treatment prompt template”).

The set of treatment instructions may be pre-stored in the database 214. The set of treatment instructions may include instructions to provide precision (or accurate) treatment pathway 220 for the query patient case based on the information from the query patient case 216, the precision diagnosis 218, and the second set of case twins. In an embodiment, the set of treatment instructions may be provided in a question-answer format (i.e., labelled data) that guides the GenAI model to provide the precision treatment pathway 220 in the same format as the treatment pathway of each of the second set of case twins.

Further, the precision treatment module 210 may add the query patient case 216, the determined precision diagnosis 218, and the second set of case twins to the set of instructions to obtain the precision treatment generation prompt. Further, the precision treatment module 210 may send the precision treatment generation prompt to the GenAI module 212. Further, the GenAI module 212 may generate a response to the treatment generation prompt using the GenAI model. The response may include the precision treatment pathway 220 for the query patient case 216. Upon generating the response, the GenAI module 212 may send the response to the precision treatment module 210. Further, the precision treatment module 210 may present the precision treatment pathway 220 on the UI 202. Used herein the “precision treatment pathway 220” refers to a specific workflow (or protocol) within a healthcare or medical application with customized treatment plans tailored to a patient's profile. In some embodiments, the precision treatment module 210 may provide recommendation of treatments for the particular patient case based on the precision treatment pathway 220. In some other embodiments, the healthcare assistance device 102 may provide the precision treatment pathway 220 to the external devices 112 (not shown). The external devices 112 may act as a Recommendation System (RS) that may recommend treatments that had positive outcomes for the patients with similar symptoms.

It should be noted that all such aforementioned modules 204-214 may be represented as a single module or a combination of different modules. Further, as will be appreciated by those skilled in the art, each of the modules 204-214 may reside, in whole or in parts, on one device or multiple devices in communication with each other. In some embodiments, each of the modules 204-214 may be implemented as dedicated hardware circuit comprising custom application-specific integrated circuit (ASIC) or gate arrays, off-the-shelf semiconductors such as logic chips, transistors, or other discrete components. Each of the modules 204-214 may also be implemented in a programmable hardware device such as a field programmable gate array (FPGA), programmable array logic, programmable logic device, and so forth. Alternatively, each of the modules 204-214 may be implemented in software for execution by various types of processors (e.g., processor 104). An identified module of executable code may, for instance, include one or more physical or logical blocks of computer instructions, which may, for instance, be organized as an object, procedure, function, or other construct. Nevertheless, the executables of an identified module or component need not be physically located together but may include disparate instructions stored in different locations which, when joined logically together, include the module and achieve the stated purpose of the module. Indeed, a module of executable code could be a single instruction, or many instructions, and may even be distributed over several different code segments, among different applications, and across several memory devices.

As will be appreciated by one skilled in the art, a variety of processes may be employed for generating precision diagnosis and precision treatment pathways based on patient case twins. For example, the exemplary system 100 and the associated healthcare assistance device 102, may generate precision diagnosis and precision treatment pathways based on patient case twins, by the processes discussed herein. In particular, as will be appreciated by those of ordinary skill in the art, control logic and/or automated routines for performing the techniques and steps described herein may be implemented by the system 100 and the associated healthcare assistance device 102 either by hardware, software, or combinations of hardware and software. For example, suitable code may be accessed and executed by the one or more processors on the system 100 to perform some or all of the techniques described herein. Similarly, application specific integrated circuits (ASICs) configured to perform some or all of the processes described herein may be included in the one or more processors on the system 100.

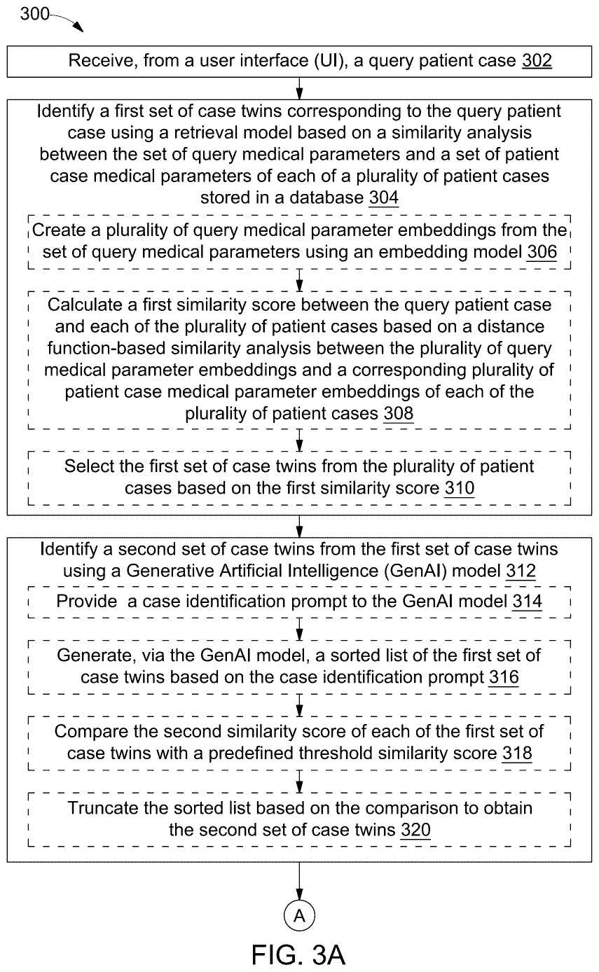

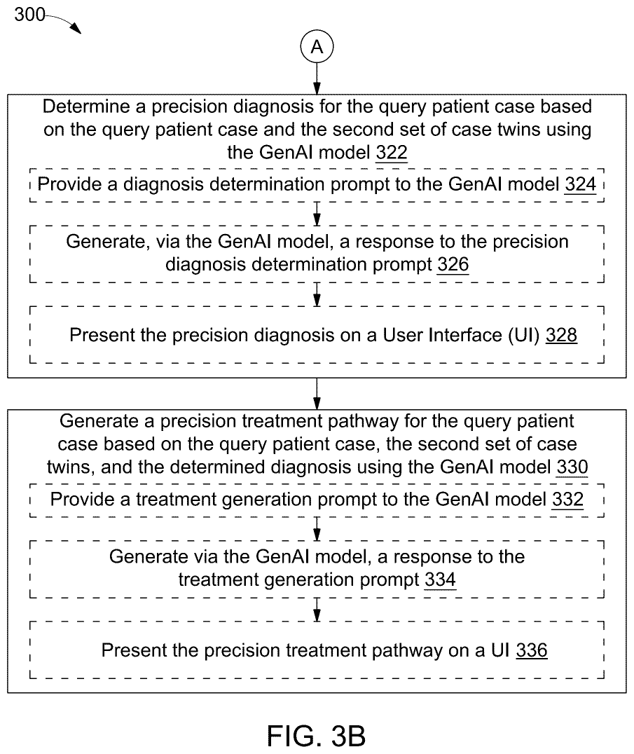

Referring now to FIGS. 3A and 3B, an exemplary process 300 for generating precision diagnosis and precision treatment pathways based on patient case twins is illustrated via a flow chart, in accordance with some embodiments of the present disclosure. The process 300 may be implemented by the healthcare assistance device 102 of the system 100. In some embodiments, the process 300 may include receiving, by a case twin retrieval module (such as the case twin retrieval module 204) a query patient case from a user device, at step 302. The query patient case may include a set of query medical parameters corresponding to a patient. The set of query medical parameters may include demographic information, medical history, symptoms, physical examination information, and laboratory test results. The query patient case may be received in the form of PDF from a user through a UI (such as the UI 202). By way of an example, the user interface may be a text-based UI or a voice-based UI. In some embodiments, the query patient case may include multi-modal data (e.g., text, images, or combination thereof).

Further, the process 300 may include identifying, by the case twin retrieval module, a first set of case twins corresponding to the query patient case using a retrieval model based on a similarity analysis between the set of query medical parameters and a set of patient case medical parameters of each of a plurality of patient cases stored in a database (such as the database 214), at step 304. The first set of case twins may include a set of similar patient cases to the query patient case. The set of similar patient cases is a subset of a plurality of patient cases stored in the database. It should be noted that each of the plurality of patient cases may include a set of patient case medical parameters, patient case diagnosis, and patient case treatment pathways. The set of patient case medical parameters may include demographic information, medical history, symptoms, physical examination information, and laboratory test results.

The step 304 may include steps 306, 308, and 310. The process 300 may include creating, by the case twin retrieval module, a plurality of query medical parameter embeddings from the set of query medical parameters using an embedding model, at step 306. Further, the process 300 may include calculating, by the case twin retrieval module, a first similarity score between the query patient case and each of the plurality of patient cases based on a distance function-based similarity analysis between the plurality of query medical parameter embeddings and a corresponding plurality of patient case medical parameter embeddings of each of the plurality of patient cases, at step 308. It should be noted that the plurality of patient case medical parameter embeddings is pre-stored in the database. Further, the process 300 may include selecting, by the case twin retrieval module, the first set of case twins from the plurality of patient cases based on the first similarity score, at step 310. Thereafter, the process 300 may proceed to step 312.

The process 300 may include identifying, by a re-assessment module (such as the re-assessment module 206), a second set of case twins from the first set of case twins using a GenAI mode, at step 312. The step 312 may include steps 314, 316, 318, and 320. The process 300 may include providing, by the re-assessment module, a case identification prompt to the GenAI model, at step 314. The case identification prompt may include the first set of case twins, the set of query medical parameters, and a set of instructions corresponding to the second set of case twins.

Further, the process 300 may include generating, by the re-assessment module, a sorted list of the first set of case twins based on the case identifications prompt, at step 316. The sorted list may include a patient case ID of each of the first set of patent cases and a second similarity score between the query patient case and each of the first set of patent cases. The second similarity score is calculated by the GenAI model. The first set of case twins in the sorted list is arranged based on the second similarity score. By way of an example, the sorted list may be arranged in such a way that the first case in the sorted list may represent the most similar patient case from the query patient case. On the other hand, the last case in the sorted list may represent the least similar patient case from the query patient case.

Further, the process 300 may include comparing, by the re-assessment module, the second similarity score of each of the first set of case twins with a predefined threshold similarity score, at step 318. The predefined threshold similarity score may be pre-configurable by the user. It should be noted that the predefined threshold similarity score may be pre-stored in the database. Further, the process 300 may include truncating, by the re-assessment module, the sorted list based on the comparison to obtain the second set of case twins, at step 320.

Thereafter, the process 300 may proceed to step 322. The process 300 may include determining, by a precision diagnosis module (such as the precision diagnosis module 208), a precision diagnosis for the query patient case based on the query patient case and the second set of case twins using the GenAI model, at step 322. The step 322 may include steps 324, 326, and 328. The process 300 may include providing, by the precision diagnosis module, a diagnosis determination prompt to the GenAI model, at step 324. The precision diagnosis determination prompt may include the set of query medical parameters, the second set of case twins, and a set of diagnosis instructions.

Further, the process 300 may include generating, by the precision diagnosis module, a response to the precision diagnosis determination prompt, at step 326. The response may include the precision diagnosis for the query patient case. Further, the process 300 may include presenting, by the precision diagnosis module, the precision diagnosis on the UI, at step 328.

Thereafter, the process 300 may proceed to step 330. The process 300 may include generating, by a precision treatment module (such as the precision treatment module 210), a precision treatment pathway for the query patient case based on the query patient case, the second set of case twins, and the determined precision diagnosis using the GenAI model, at step 330. The step 330 may include steps 332, 334, and 336.

The process 300 may include providing, by the precision treatment module, a treatment generation prompt to the GenAI model, at step 332. The treatment generation prompt may include the set of query medical parameters, the second set of case twins, and a set of treatment instructions. Further, the process 300 may include generating, by the precision treatment module via using the GenAI model, a response to the treatment generation prompt, at step 334. The response may include the precision treatment pathway for the query patient case. Further, the process 300 may include presenting, by the precision treatment module, the precision treatment pathway on the UI, at step 336.

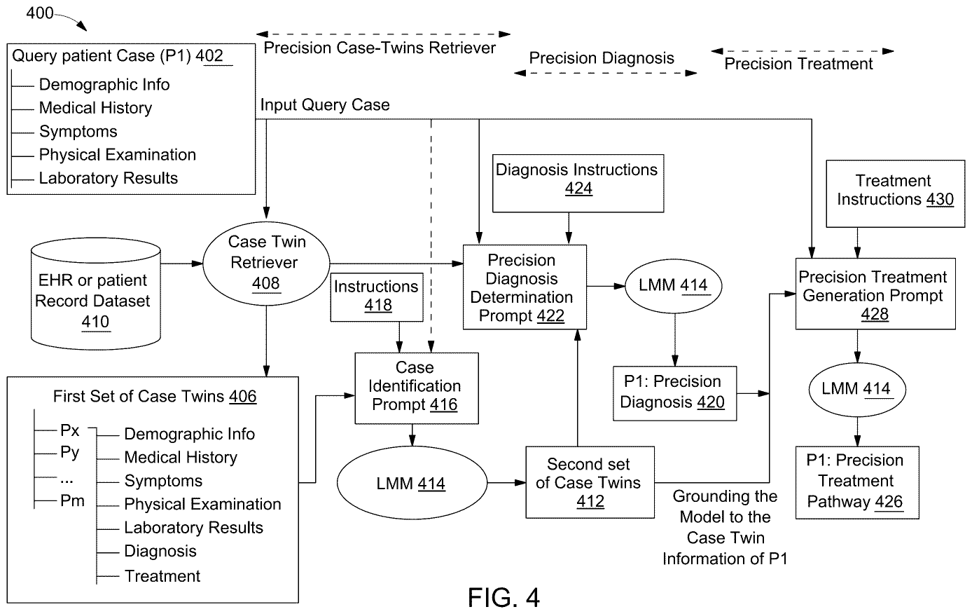

Referring now to FIG. 4, a detailed exemplary process 400 for generating precision diagnosis and precision treatment pathway based on patient case twins is depicted via a flowchart, in accordance with an embodiment of the present disclosure. FIG. 4 is explained in conjunction with FIGS. 1, 2, and 3. The process 400 may be implemented by the healthcare assistance device 102. Initially, the process 400 may include receiving, by the case twin retrieval module 204, a query patient case 402 corresponding to a patient from a user (such as a clinician, etc.) through a UI (such as the UI 202). The query patient case 402 may include one or more documents.

The query patient case 402 may include information (i.e., a set of query medical parameters) corresponding to the patient. By way of an example, the set of query medical parameters may include, but may not be limited to, demographic information, medical history, symptoms, physical examination, and laboratory test results. In some embodiments, the set of query medical parameters may also include multi-model database (or information) (e.g., any imaging data such as, MRI) corresponding to the patient. Additionally, the set of query medical parameters may include a patient ID corresponding to the patient. By way of an example, the patient ID for the query patient case 402 may be ‘P1’.

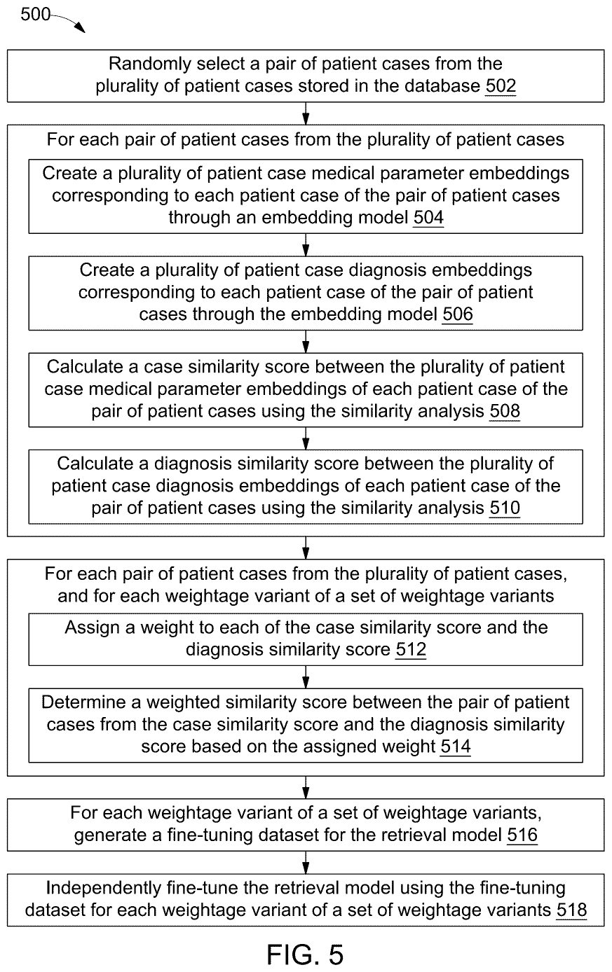

Further, the process 400 may include identifying, by the case twin retrieval module 204, a first set of case twins 406 corresponding to the query patient case 402 using a case twin retriever 408 (i.e., a retrieval model). The case twin retriever 408 may identify the first set of case twins from a plurality of patient cases pre-stored in an EHR 410 (or a patient record database). Each of the plurality of patient cases may include a set of patient case medical parameters, a patient case diagnosis, and a patient case treatment pathway. The case twin retriever 408 may be fine-tuned using last layer representation (obtained from the case twin retriever 408) of the plurality of patient cases stored in the EHR) 410. This is further explained in greater detail in conjunction with FIGS. 5, 6, and 7.

The case twin retriever 408 may identify the set of case twins based on a similarity analysis between the set of query medical parameters and the set of patient case medical parameters of each of the plurality of patient cases. The set of patient case medical parameters may correspond to the set of query medical parameters. The set of patient case medical parameters may include demographic information, medical history, symptoms, physical examination information, and laboratory test results.

To identify the first set of case twins 406, the case twin retrieval module 204, may create a plurality of query medical parameter embeddings (i.e., dense embeddings) from the set of query medical parameters using an embedding model. Further, the case twin retrieval module 204, via the case twin retriever 408, may extract (or retrieve) the plurality of query medical parameter embeddings. Further, the case twin retrieval module 204, via the case twin retriever 408, may retrieve the plurality of patient case medical parameter embeddings (i.e., dense embeddings) from the EHR 410. The plurality of patient case medical parameter embeddings from the EHR 410 may be pre-extracted by the case twin retriever 408.

Further, the case twin retrieval module 204 may calculate a first similarity score between the query patient case 402 and each of the plurality of patient cases based on a distance function-based similarity analysis (e.g., a cosine similarity) between the plurality of query medical parameter embeddings and a corresponding plurality of patient case medical parameter embeddings of each of the plurality of patient cases. In an embodiment, a cosine distance function may be used to calculate the distance of the query patient case with each of the plurality of patient cases in the EHR 410 in the embedding space utilizing their dense embeddings. By way of an example, the first similarity score may be calculated using a formula (1).

Similarity Score=1−(distance)

Further, the case twin retrieval module 204 may identify the first set of case twins 406 from the plurality of patient cases based on the first similarity score. The number (or range) for the first set of closest case twins may be pre-defined or pre-configurable by the user. The case twin retrieval module 204 may select the number of patient cases from the plurality of patient cases having a similarity score higher than remaining of the plurality of patient cases. By way of an example, the plurality of patient cases includes ‘n’ number of patient cases in the EHR 410. The predefined number for the first set of case twins 406 may be defined as ‘m’ (where m<n). Thus, for the first set of case twins 406, the case twin retrieval module 204 may select top ‘m’ patient cases from the ‘n’ patient cases in decreasing order of the first similarity score.

In an alternative embodiment, the user may provide a predefined threshold similarity score for identifying the first set of case twins 406. The predefined threshold similarity score may indicate a degree of similarity to the query patient case required by the user. In such an embodiment, the case twin retrieval module 204 may select patient cases from the plurality of patient cases having a similarity score higher than the predefined threshold similarity score. By way of an example, the plurality of patient cases includes ‘n’ number of patient cases in the EHR 410. The predefined threshold similarity score for the first set of case twins 406 may be defined as ‘x’. Thus, for the first set of case twins 406, the case twin retrieval module 204 may select patient cases from the ‘n’ patient cases for which the first similarity score is higher than ‘x’. If ‘m’ number of patient cases have the first similarity score higher than ‘x’, then the first set of case twins 406 may include ‘m’ patient cases.

Once the first set of case twins 406 is selected, the case twin retrieval module 204 may retrieve the first set of case twins 406 from the EHR 410 with associated information (such as the set of patient case medical parameters (e.g., patient ID, demographic information, medical history, symptoms, physical examination information, laboratory test results, etc.), patient case diagnosis, and patient case treatment pathway). By way of an example, for a query patient case (patient ID ‘P1’), ‘m’ number of patient cases may be identified in the first set of case twins. The patient IDs of the first set of case twins may be ‘Px’, ‘Py’, . . . , ‘Pm’. Further, the case twin retrieval module 204 may send the first set of case twins to the re-assessment module 206.

Further, the process 400 may include identifying, by the re-assessment module 206 through the GenAI module 212, a second set of case twins 412 from the first set of case twins 406 using an LMM 414 (analogous to the GenAI model). To identify the second set of case twins 412, the re-assessment module 206 may construct a case identification prompt 416 using a set of instructions 418 (i.e., a re-assessment prompt template). The set of instructions 418 may be pre-stored in the database 214. The case identification prompt 416 may include the query patient case 402, the first set of case twins 406, and the set of instructions 418. The set of instructions 418 may include instructions to provide the first similarity score between the query patient case 402 and each case twin within the first set of case twins 406. In an embodiment, the set of instructions 418 may include instructions for the LMM 414 to keep the first similarity score between the range of 0 to 1. In such an embodiment, the first similarity score of 0 for a patient case may indicate that the patient case is completely different from the query patient case 402. Similarly, the first similarity score of 1 for a patient case may indicate that the patient case is completely similar to the query patient case 402. The set of instructions 418 may also include instructions to provide a sorted list (or an ordered list) of the first set of case twins 406 based on a second similarity score (determined by the LMM 414).

By way of an example, an exemplary template of the case identification prompt 416 may be described as below.
“I want you to act like a retrieval model. You will be provided with a patient information and a set of other patients' information. Your task is to calculate the similarity score between the given query patient with the other patients in the range from 0 to 1, where 1 representing the most similar patient and 0 representing the least similar patient. Next, please order the given set of other patients based on the similarity scores where the first patient in the list represents the most similar patient with the highest similarity score.
Here is a query patient P: [CASE_INPUT]
Set of other patients:

$$P 1 : [ P 1 CASE\_INPUT ]$$

$$P 2 : [ P 2 CASE\_INPUT ]$$

$$…$$

$$P m : [ P m CASE\_INPUT ]$$

The response must be brief and follow this format: [(Patient ID: similarity score), (Patient ID: similarity score) . . . ].
Now, please provide the ordered list of patients along with their similarity scores as per the above instructions.”

When the case identification prompt is constructed using the above mentioned prompt template by the re-assessment module 206, ‘[CASE_INPUT]’ may be replaced with the set of query medical parameters (i.e., demographics, medical history, symptoms, physical examination, and laboratory results information) of the query patient case. Additionally, ‘[P1 CASE_INPUT]’, ‘[P2 CASE_INPUT]’, . . . , ‘[Pm CASE_INPUT]’ may be replaced with the set of patient case medical parameters (i.e., patient ID, demographics, medical history, symptoms, physical examination, and laboratory results information) of each of the first set of case twins.

The re-assessment module 206 may then send the case identification prompt 416 to the GenAI module 212. The GenAI module 212 may input the case identification prompt 416 to the LMM 414. The second similarity score is then calculated by the LMM 414 and provided as an output in the sorted list. The sorted list may include a patient case ID (e.g., numeric value) of each of the first set of patient cases and the second similarity score between the query patient case 402 and each of the first set of case twins 406. It should be noted that the first set of case twins 406 in the sorted list is arranged based on the second similarity score. The first patient case (i.e., topmost patient case) in the sorted list may represent the most similar patient case to the query patient case 402 among the first set of case twins. On the other hand, the last patient case (i.e., bottom-most patient case) in the sorted list may represent the least similar patient case to the query patient case 402 among the first set of case twins. Once the sorted list is generated, the GenAI module 212 may send the sorted list to the re-assessment module 206. The sorted list may be received in the form of tabular format. In an embodiment, the table may include two columns-One column for the patient ID and another column for the second similarity score.

Further, the re-assessment module 206 may compare the second similarity score of each of the first set of case twins 406 with a predefined threshold similarity score (e.g., 0.9, 90%, etc.). The predefined threshold similarity score may be configurable by the user. Further, the re-assessment module 206 may truncate the sorted list based on the comparison to obtain the second set of case twins 412. In other words, the sorted list may be truncated using the predefined threshold similarity score as a limit. The patient cases having the second similarity score above the predefined threshold similarity score in the sorted list are selected as the second set of case twins 412. Further, the re-assessment module 206 may send the second set of case twins 412 to the precision diagnosis module 208.

In continuation of the above example, the re-assessment module 206 may identify the second set of case twins 412 the ‘m’ patient cases in the first set of case twins 406 using the LMM 414. The GenAI module 212 may calculate the second similarity score for each of the ‘m’ patient cases using the LMM 414. Further, the GenAI module 212 may generate, via the LMM 414, a sorted list which may include the ‘m’ patient cases arranged in a descending order of the second similarity score. Further, the GenAI module 212 may truncate the sorted list to include patient cases for which the second similarity score is above the predefined threshold score. For example, ‘k’ number of patient cases may be identified as the second set of case twins (where k<m).

Further, the process 400 may include generating, by the precision diagnosis module 208, a precision diagnosis 420 for the query patient case 402. The precision diagnosis module 208 may determine the precision diagnosis 420 for the query patient case 402 based on the query patient case 402 and the second set of case twins 412, through the GenAI module 212 using the LMM 414. To determine the precision diagnosis 420, the precision diagnosis module 208 may construct a precision diagnosis determination prompt 422 using a set of diagnosis instructions 424 (i.e., a precision diagnosis prompt template). The set of diagnosis instructions 424 may be pre-stored in the database 214. The set of diagnosis instructions 424 may include instructions for the LMM 414 to provide the precision (or accurate) diagnosis 420 for the query patient case 402 based on the information received from the query patient case 402 (i.e., the set of query medical parameters) and the information from the second set of case twins 412 (i.e., the set of patient case medical parameters and the patient case diagnosis). In an embodiment, the set of diagnosis instructions 424 may be in a question-answer format that may guide the LMM 414 to provide the precision diagnosis 420 in the same format as the format of the patint case diagnosis in each of the second set of case twins 412. The precision diagnosis module 208 may create the precision diagnosis determination prompt 422 by adding the set of query medical parameters and the second set of case twins 412 to the set of diagnosis instructions 424.

By way of an example, an exemplary precision diagnosis prompt template is described as below.
“I want you to act like a professional clinician. You will diagnose a patient's health condition based on provided information such as demographics, medical history, symptoms, physical examination, and laboratory results.
In addition, a few similar case examples are provided to aid in diagnosis.
Example Case 1: [Example 1 CASE_INPUT]
Which medical condition or disease patient is suffering from? please diagnose the given case, a short factoid diagnosis, often between 1 and 9 words.
Answer: [Example 1 Diagnosis]
Example Case 2: [Example 2 CASE_INPUT]
Which medical condition or disease patient is suffering from? please diagnose the given case, a short factoid diagnosis, often between 1 and 9 words.
Answer: [Example 2 Diagnosis]
. . .
Example Case k: [Example k CASE_INPUT]
Which medical condition or disease patient is suffering from? please diagnose the given case, a short factoid diagnosis, often between 1 and 9 words.
Answer: [Example k Diagnosis]
Here is a query case: [CASE_INPUT]
Which medical condition or disease patient is suffering from? please diagnose the given case, a short factoid diagnosis, often between 1 and 9 words.
Answer:”

When the precision diagnosis prompt is constructed using the precision diagnosis prompt template, ‘[Example 1 CASE_INPUT]’, ‘[Example 2 CASE_INPUT]’, . . . , ‘[Example k CASE_INPUT]’ are replaced with the set of patient case medical parameters of the respective patient cases from the second set of case twins 412. Additionally, ‘[Example 1 Diagnosis]’, ‘[Example 2 Diagnosis]’, . . . , ‘[Example k Diagnosis]’ are replaced with diagnosis information of the respective patient cases from the second set of case twins 412. Additionally, ‘[CASE_INPUT]’ is replaced with the set of query medical parameters of the query patient case 402.

Further, the precision diagnosis module 208 may provide the precision diagnosis determination prompt 422 to the GenAI module 212. The GenAI module 212 may then input the precision diagnosis determination prompt 422 to the LMM 414. Further, the GenAI module 212 may generate the precision diagnosis 420 for the query patient case 402 using the LMM 414 in response to the precision diagnosis determination prompt 422. Further, the GenAI module 212 may send the precision diagnosis 420 for the query patient case 402 to the precision diagnosis module 208. Further, the precision diagnosis module 208 may present the precision diagnosis 420 on a UI (such as the UI 202). Further, the precision diagnosis module 208 may send the precision diagnosis 420 to the precision treatment module 210.

Further, the process 400 may include generating, by the precision treatment module 210, a precision treatment pathway 426 for the query patient case 402. The precision treatment module 210 may generate the precision treatment pathway 426 based on the query patent case 402, the second set of case twins 412, and the determined precision diagnosis 420 using the LMM 414. To determine the precision treatment pathway 426, the precision treatment module 210 may construct a precision treatment generation prompt 428 using a set of treatment instructions 430 (i.e., a precision treatment prompt template). The set of treatment instructions 430 may be pre-stored in the database 214.

The set of treatment instructions 430 may include instructions for the LMM 414 to provide the precision (or accurate) treatment pathway 426 for the query patient case 402 based on the information received from the query patient case 402 (i.e., the set of query medical parameters), the information from the second set of case twins 412 (i.e., the set of medical parameters and the patient case diagnosis, and the patient case treatment pathway), and the precision diagnosis 420. By way of an example, the precision treatment generation prompt 428 may be provided in a question-answer format that may guide the LMM 414 to provide the precision treatment pathway 426 in the same format as the format of the patient case treatment pathway.

The precision treatment module 210 may create the precision treatment generation prompt 428 by adding the set of query medical parameters, the second set of case twins 412, and the determined precision diagnosis 420.

By way of an example, an exemplary precision treatment prompt template is described as below.
“I want you to act like a professional clinician. You will provide a precise treatment plan for a patient's health condition based on provided information such as demographics, medical history, symptoms, physical examination, and laboratory results, followed by diagnosis information.
In addition, a few similar case examples are provided to aid in constructing a precise treatment plan.
Example Case 1: [Example 1 CASE_INPUT]
Diagnosis - - - [Example 1 Diagnosis]
What would be a precise treatment plan for this patient, including invasive/non-invasive procedures and medications? Please provide a precise treatment plan for the given case, a short factoid plan, often between 1 and 5 lines.
Answer: [Example 1 Treatment]
Example Case 2: [Example 2 CASE_INPUT]
Diagnosis - - - [Example 2 Diagnosis]
What would be a precise treatment plan for this patient, including invasive/non-invasive procedures and medications? Please provide a precise treatment plan for the given case, a short factoid plan, often between 1 and 5 lines.
Answer: [Example 2 Treatment]
. . .
Example Case k: [Example k CASE_INPUT]
Diagnosis - - - [Example k Diagnosis]
What would be a precise treatment plan for this patient, including invasive/non-invasive procedures and medications? Please provide a precise treatment plan for the given case, a short factoid plan, often between 1 and 5 lines.
Answer: [Example k Treatment]
Here is a query case: [CASE_INPUT]
Diagnosis - - - [DIAGNOSIS]
What would be a precise treatment plan for this patient, including invasive/non-invasive procedures and medications? Please provide a precise treatment plan for the given case, a short factoid plan, often between 1 and 5 lines.
Answer:”

When the precision treatment generation prompt 428 is constructed using the above prompt template, ‘[Example 1 CASE_INPUT]’, ‘[Example 2 CASE_INPUT]’, . . . , ‘[Example k CASE_INPUT]’ are replaced with the set of query medical parameters of the respective patient cases from the second set of case twins 412. Additionally, ‘[Example 1 Diagnosis]’, ‘[Example 2 Diagnosis]’, . . . , ‘[Example k Diagnosis]’ are replaced with the patient case diagnosis of the respective patient cases from the second set of case twins 412. Additionally, ‘[Example 1 Treatment]’, [Example 2 Treatment]′, . . . , ‘[Example k Treatment]’ are replaced with the patient case treatment pathway of the respective patient cases from the second set of case twins 412. Additionally, ‘[CASE_INPUT]’ is replaced with the set of query medical parameters for the query patient case 402. Additionally, ‘[DIAGNOSIS]’ is replaced with the precision diagnosis 420 for the query patient case 402 obtained through the precision diagnosis module 208.

Once the precision treatment generation prompt 428 is created, the precision treatment module 210 may provide the precision treatment generation prompt 428 to the GenAI module 212. Further, the GenAI module 212 may generate the precision treatment pathway 426 for the query patient case 402 in response to the precision treatment generation prompt 428. Further, the precision treatment module 210 may present the precision treatment pathway 426 on a UI (such as the UI 202).

Referring now to FIG. 5, an exemplary process 500 for fine tuning retrieval models (such as the case twin retriever 408) is depicted via a flowchart, in accordance with an embodiment of the present disclosure. FIG. 5 is explained in conjunction with FIGS. 1, 2, 3, and 4. The process 500 may be implemented by the healthcare assistance device 102. To fine-tune a retrieval model, a fine-tuning dataset may be created from a plurality of patient cases retrieved from a database (such as the database 214 or the EHR 410. The process 500 may include randomly selecting, by a case twin retrieval module (such as the case twin retrieval module 204), a pair of patient cases from the plurality of patient cases stored in a database, at step 502.

Further, for each pair of patient cases from the plurality of patient cases, the process 500 may include creating, by the case twin retrieval module, a plurality of patient case medical parameter embeddings corresponding to each patient case of the pair of patient cases through an embedding model, at step 504. The embedding model may be, for example, but may not be limited to, Word2Vec, Glove, or BERT.

Further, the process 500 may include creating, by the case twin retrieval module, a plurality of patient case diagnosis embeddings corresponding to each patient case of the pair of patient cases through the embedding model, at step 506. In some embodiments, the creation of the plurality of patient case medical parameter embeddings may be simultaneous to the creation of the plurality of patient case diagnosis embeddings by the case twin retrieval module. In some other embodiments, the case twin retrieval module may sequentially create the plurality of patient case medical parameter embeddings and the plurality of patient case diagnosis embeddings.

Further, the process 500 may include calculating, by the case twin retrieval module, a case similarity score between the plurality of patient case medical parameter embeddings of each patient case of the pair of patient cases using a similarity analysis (for example, cosine similarity analysis), at step 508.

Further, the process 500 may include calculating, by the case twin retrieval module, a diagnosis similarity score between the plurality of patient case diagnosis embeddings of each patient case of the pair of patent cases using the similarity analysis, ats step 510.

Further, for each pair of patient cases from the plurality of patient cases, and for each weightage variant of a set of weightage variants, the process 500 may include assigning, by the case twin retrieval module, a weight to each of the case similarity score and the diagnosis similarity score, at step 512. It should be noted that the weight is predefined for the weightage variant. By way of an example, a weightage variant may include 25% weight assigned to the case similarity score and 75% weight assigned to the diagnosis similarity score for a pair of patient cases. This is discussed in greater detail in conjunction with FIGS. 6 and 7.

Further, the process 500 may include determining, by the case twin retrieval module, a weighted similarity score between the pair of patient cases from the case similarity score and the diagnosis similarity score based on the assigned weight, at step 514. It should be noted that the weighted similarity score is a weighted average of the case similarity score and the diagnosis similarity score based on the assigned weight.

Further, for each weightage variant of a set of weightage variants, the process 500 may include generating, by the case twin retrieval module, a fine-tuning dataset for the retrieval model, at step 516. It should be noted that the fine-tuning dataset may include each of the plurality of patient cases and the associated weighted similarity score between each pair of patient cases from the plurality of patient cases for the weightage variant.

Further, the process 500 may include independently fine-tuning, by the case twin retrieval module, the retrieval model using the fine-tuning dataset for each weightage variant of a set of weightage variants, at step 518.

Referring now to FIG. 6, a detailed exemplary process 600 for fine-tuning retrieval models is depicted via a flowchart, in accordance with some embodiments of the present disclosure. FIG. 6 is explained in conjunction with FIGS. 1, 2, 3, 4, and 5. The case twin retrieval module 204 may fine-tune a base retrieval model 602 (e.g., clinicalBERT) using a large set of patient records (i.e., the plurality of patient cases). The large set of patient records may be pre-stored in a patient record dataset 604 (analogous to the EHR 410) within the database 214. In some embodiments, an EHR (such as the EHR 410) may not be available due to various reasons, such as regulatory guidelines to protect the patient information and/or lack of infrastructure to precure and manage the set of patient records in a centralized system. In such embodiments, one or more repositories (for example, PubMed Central (PMC), MEDLINE, PubMed, and the like) may be used to prepare the patient record dataset 604. The repository may include millions of patient case studies 606, including those with commercial licenses.

Each of the repository case studies 606 may present specific patients or a group of patients, highlighting their associated clinical characteristics, challenges, the patient case diagnosis, and the patient case treatment pathway provided. Each of the repository case studies 606 may also include the set of patient case medical parameters (i.e., demographic information, medical history, symptoms, physical examination information, and laboratory test results).

Further, the case twin retrieval module 204 may download the repository case studies 606. The repository case studies 606 may be in a format of, for example, but may not be limited to, PDF, word document (DOC or DOCX), and database records. The repository case studies 606 may then be converted into text (TXT) format using a file to text conversion algorithm (such as a PDF to Text converter 608).

Further, the case twin retrieval module 204 may send the repository case studies 606 to an LMM (such as the LMM 414). Further, the LMM may perform extraction of multimodal data from the repository case studies 606, at step 610. The case twin retrieval module 204 may send a prompt 612A to the LMM including instructions for multimodal data extraction. Further, the case twin retrieval module 204 may send the extracted multimodal data to an LLM (or the same LMM as the one used in the step 610). The LLM may perform classification of the multimodal data, at step 614. The case twin retrieval module 204 may send a prompt 612B to the LLM including instructions for parsing each of the repository case studies 606 and segmenting the multimodal data into categories such as the set of patient case medical parameters (i.e., demographic information, medical history, symptoms, physical examination information, and laboratory test results), diagnosis, and treatment. It should be noted that each of the repository case studies 606 may be assigned a patient ID (such as P1, P2, P3, and so on). Further, the case twin retrieval module 204 may standardize the segmented data, at step 616. Further, upon standardization, the set of case medical parameters may be stored in the patient record dataset 604.

In an embodiment, the case twin retrieval module 204 may randomly select a subset dataset 618 from the patient record dataset 604 to fine-tune the retrieval model 602. Further, from the subset dataset 618, the case twin retrieval module 204 may randomly select ‘s’ number of pairs of patient cases. It should be noted that ‘s’ is configurable by the user (and may be recommended to be greater than 100,000). The subset dataset 618 may include two columns. A first column may correspond to a first patient case 618A from the pair of patient cases, and another column may correspond to a second patient case 618B from the pair of patient cases.

By way of an example, for a first pair of patient cases, the patient ID of the first patient case 618A may be ‘P1’, and the patient ID of the second patient case 618B may be ‘P2’. For a second pair of patient cases, the patient ID of the first patient case 618A may be ‘P1’, and the patient ID of the second patient case 618B may be ‘P5’. For a third pair of patient cases, the patient ID of the first patient case 618A may be ‘P2’, and the patient ID of the second patient case 618B may be ‘P4’.

Upon randomly selecting the pair of patient cases, the case twin retrieval module 204 may construct a fine-tuning dataset 620 through step 622 of training data construction based on a similarity context strategy 624. The similarity context strategy 624 may include training data construction based on a set of weightage variants. The fine-tuning dataset 620 may be created to fine-tune the retrieval model 602 for more accurate identification of the first set of case twins. In an embodiment, the set of weightage variants may include a pre-diagnosis-only, a diagnosis-lite, a diagnosis-intensive, a diagnosis-dominant, and a diagnosis-only weightage variant. This is explained in greater detail in conjunction with FIG. 7.

Referring now to FIG. 7, a detailed exemplary process 700 for training data construction based on predefined weightage variants is depicted via a flowchart, in accordance with an embodiment of the present disclosure. FIG. 7 is explained in conjunction with FIGS. 2, 5, and 6. As will be appreciated, identification of relevant case twins depends not just on the similarity of pre-diagnosis patient information (or the set of patient case medical parameters, such as demographic details and medical history) but also on the patient case diagnosis. Two patient cases are deemed case twins of each other if they share similar diagnosis too. For example, in a case of two patient cases, both the patients are of the same gender, belong to the same age group, are diabetic, and have high cholesterol levels. In such a case, some clinicians may find a similarity between the two patient cases even if the diagnoses for the two patient cases are different. Similarly, in a case of two patient cases, both the patients may be diagnosed with a rare disease (such as Progressive multifocal leukoencephalopathy (PML)). In such a case, a clinician may be interested in the other patient case (finding similarity on grounds of common diagnoses) even if the pre-diagnosis medical information of the two patients is different.

Thus, the retrieval model 602 may be fine-tuned based on different weightage variants of datasets, each assigning different weights (i.e., importance) to case similarity scores (or medical parameter similarity scores) and diagnosis similarity scores of the pairs of patient cases.

Once the pair of patient cases is randomly selected, for each pair of patient cases from the plurality of patient cases, the case twin retrieval module 204 may create a plurality of patient case medical parameter embeddings corresponding to each patient case of the pair of patient cases through the retrieval model 602 (i.e., an embedding model (e.g., ClinicalBERT)). Additionally, the case twin retrieval module 204 may create a plurality of patient case diagnosis embeddings corresponding to each patient case of the pair of patient cases through the embedding model. In some embodiments, the creation of the plurality of patient case medical parameter embeddings may be simultaneous to the creation of the plurality of patient case diagnosis embeddings. In some other embodiments, the case twin retrieval module 204 may sequentially create the plurality of patient case medical parameter embeddings and the plurality of patient case diagnosis embeddings.

Further, the case twin retrieval module 204 may calculate a case similarity score between the plurality of patient case medical parameter embeddings of each patient case of the pair of patient cases using the similarity analysis (e.g., cosine similarity analysis). Additionally, the case twin retrieval module 204 may calculate a diagnosis similarity score between the plurality of patient case diagnosis embeddings of each patient case of the pair of patient cases using the similarity analysis.

Further, for each of patient cases from the plurality of patient cases, and for each weightage variant of a set of weightage variants, the case twin retrieval module 204 may assign a weight to each of the case similarity score and the diagnosis similarity score. The weight is predefined for the weightage variant. The set of weightage variants may include a pre-diagnosis-only weightage variant, a diagnosis-lite weightage variant, a diagnosis-intensive weightage variant, a diagnosis-dominant weightage variant, and a diagnosis-only weightage variant.

By way of an example, a pair of patient cases may include patient cases corresponding to patient IDs ‘Patient-1’ and ‘Patient-2’. ‘Patient-1’ may include case information 702A and diagnosis 702B. ‘Patient-2’ may include case information 704A and diagnosis 704B. The case twin retrieval module 204 may create pre-diagnosis embeddings 706A (i.e., a plurality of patient case medical parameter embeddings) from the case information 702A through the retrieval model 602 (i.e., embedding model). Additionally, the case twin retrieval module 204 may create diagnosis embeddings 706B (i.e., a plurality of patient case diagnosis embeddings) from the diagnosis 702B through the retrieval model 602. Similarly, the case twin retrieval module 204 may create pre-diagnosis embeddings 708A from the case information 704A through the retrieval model 602. Additionally, the case twin retrieval module 204 may create diagnosis embeddings 708B from the diagnosis 704B through the retrieval model 602.

Further, the case twin retrieval module 204 may calculate a cosine pre-diagnosis similarity 710 (i.e., a case similarity score based on cosine distance) between the pre-diagnosis embeddings 706A and the pre-diagnosis embeddings 708A. Similarly, the case twin retrieval module 204 may calculate cosine diagnosis similarity 712 (i.e., a diagnosis similarity score based on cosine distance) between the diagnosis embeddings 706B and the diagnosis embeddings 708B.

Further, the case twin retrieval module 204 may assign a weight to each of the cosine pre-diagnosis similarity 710 and the cosine diagnosis similarity 712. It should be noted that the weight is predefined for the weightage variant.

By way of an example, five weightage variants may be used based on varying weights assigned to the cosine pre-diagnosis similarity 710 and the cosine diagnosis similarity 712 by varying the weight.

A pre-diagnosis-only variant 714A may assign a 100% (or 1.00) weight to the cosine pre-diagnosis similarity 710 and 0% (or 0.00) weight to the cosine diagnosis similarity 712. In other words, through the pre-diagnosis-only variant 714A, the retrieval model 602 may use only case information to identify the case twins.

A diagnosis-lite variant 714B may assign a 75% (or 0.75) weight to the cosine pre-diagnosis similarity 710 and 25% (or 0.25) weight to the cosine diagnosis similarity 712. In other words, through the diagnosis-lite variant 714B, the retrieval model 602 may use case information with weightage of 0.75 and diagnosis information with weightage of 0.25 to identify the case-twins.

A diagnosis-intensive variant 714C may assign a 50% (or 0.50) weight to the cosine pre-diagnosis similarity 710 and 50% (or 0.50) weight to the cosine diagnosis similarity 712. In other words, through the diagnosis-intensive variant 714C, the retrieval model 602 may use case information with weightage of 0.50 and diagnosis information with weightage of 0.50 to identify the case-twins.

A diagnosis-dominant variant 714D may assign a 25% (or 0.25) weight to the cosine pre-diagnosis similarity 710 and 75% (or 0.75) weight to the cosine diagnosis similarity 712. In other words, through the diagnosis-dominant variant 714D, the retrieval model 602 may use case information with weightage of 0.25 and diagnosis information with weightage of 0.75 to identify the case-twins.

A diagnosis-only variant 714E may assign a 0% (or 0.00) weight to the cosine pre-diagnosis similarity 710 and 100% (or 1.00) weight to the cosine diagnosis similarity 712. In other words, through the pre-diagnosis-only variant 714A, the retrieval model 602 may use diagnosis information only to identify the case twins.

Further, the case twin retrieval module 204 may determine a weighted similarity score between the pair of patient cases from the case similarity score and the diagnosis similarity score based on the assigned weight. The weighted similarity score is a weighted average of the case similarity score and the diagnosis similarity score based on the assigned weight.

Referring back to FIG. 6, upon determination, for each weightage variant of the set of weightage variants, the case twin retrieval module 204 may generate a fine-tuning dataset 620 for the retrieval model 602. It should be noted that the fine-tuning dataset 620 may include each of the plurality of patient cases and the associated weighted similarity score between each pair of patient cases from the plurality of patient cases for the weightage variant. In other words, the fine-tuning dataset 620 may include the pair of sentences (case information and diagnosis information) along with their associated similarity scores across the five weightage variants. In an embodiment, the fine-tuning dataset 620 may include the column for first patient case 618A in a pair, the column for the second patient case 618B in the pair, and a column for a weighted similarity score 626.

By way of an example, for the first pair of patient cases ‘P1’ and ‘P2’, the weighted similarity score 626 may be ‘0.9’. For the second pair of patient cases ‘P1’ and ‘P5’, the weighted similarity score 626 may be ‘0.2’. For the third pair of patient cases ‘P2’ and ‘P4’, the weighted similarity score 626 may be ‘0.3’. It should be noted that the weighted similarity score 626 may be calculated based on one of the 5 weightage variants.

Further, once the fine-tuning dataset 620 is generated, the case twin retrieval module 204 may independently fine-tune the retrieval model 602 using the fine-tuning dataset 620 for each weightage variant of the set of weightage variants. Further, the retrieval model 602 is fine-tuned through step 628 of fine-tuning, to obtain the case twin retriever 408.

In some embodiments, the retrieval model 602 (or the case twin retriever 408) may be evaluated based on all five weightage variants using a test patient record dataset (which may also be obtained from the patient record dataset 604). It should be noted that in the inference (i.e., deployment) phase, one of the weightage variants can be used by the case twin retriever 408 to retrieve the case twins. Alternatively, a combination of one or more of the five weightage variants can be used by the case twin retriever 408 to retrieve the case twins in the inference phase.

As will be also appreciated, the above-described techniques may take the form of computer or controller implemented processes and apparatuses for practicing those processes. The disclosure can also be embodied in the form of computer program code containing instructions embodied in tangible media, such as floppy diskettes, solid state drives, CD-ROMs, hard drives, or any other computer-readable storage medium, wherein, when the computer program code is loaded into and executed by a computer or controller, the computer becomes an apparatus for practicing the invention. The disclosure may also be embodied in the form of computer program code or signal, for example, whether stored in a storage medium, loaded into and/or executed by a computer or controller, or transmitted over some transmission medium, such as over electrical wiring or cabling, through fiber optics, or via electromagnetic radiation, wherein, when the computer program code is loaded into and executed by a computer, the computer becomes an apparatus for practicing the invention. When implemented on a general-purpose microprocessor, the computer program code segments configure the microprocessor to create specific logic circuits.

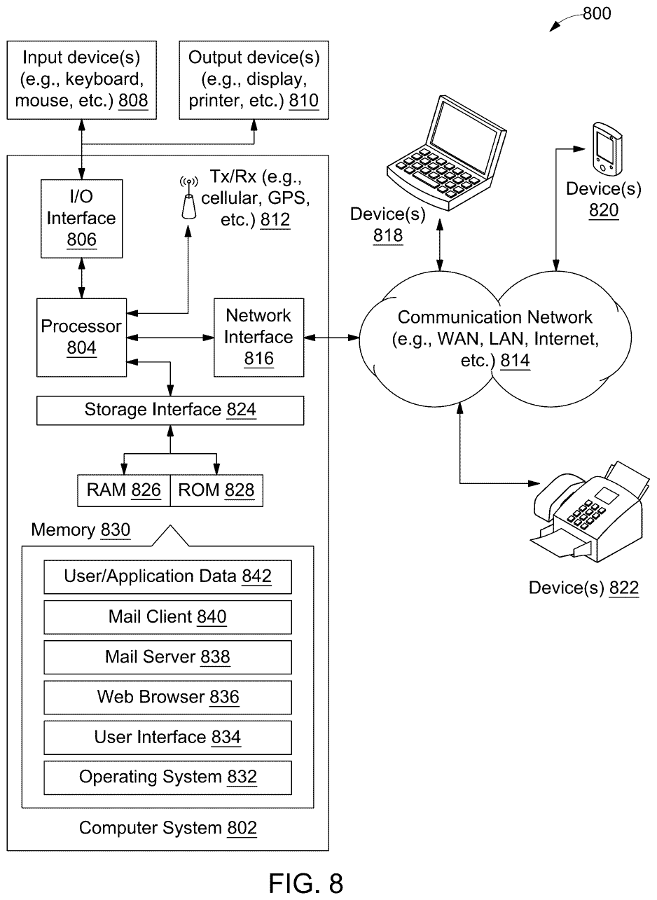

The disclosed methods and systems may be implemented on a conventional or a general-purpose computer system, such as a personal computer (PC) or server computer. Referring now to FIG. 8, a block diagram 800 of an exemplary computer system 802 for implementing embodiments consistent with the present disclosure is illustrated. Variations of computer system 802 may be used for implementing system 100 for generating precision diagnosis and precision treatment pathways based on patient case twins. The computer system 802 may include a central processing unit (“CPU” or “processor”) 804. The processor 804 may include at least one data processor for executing program components for executing user-generated or system-generated requests. A user may include a person, a person using a device such as such as those included in this disclosure, or such a device itself. The processor 804 may include specialized processing units such as integrated system (bus) controllers, memory management control units, floating point units, graphics processing units, digital signal processing units, etc. The processor 804 may include a microprocessor, such as AMD® ATHLON®, DURON® OR OPTERON®, ARM's application, embedded or secure processors, IBM® POWERPC®, INTEL® CORE® processor, ITANIUM® processor, XEON® processor, CELERON® processor or other line of processors, etc. The processor 804 may be implemented using mainframe, distributed processor, multi-core, parallel, grid, or other architectures. Some embodiments may utilize embedded technologies like application-specific integrated circuits (ASICs), digital signal processors (DSPs), Field Programmable Gate Arrays (FPGAs), etc.

The processor 804 may be disposed in communication with one or more input/output (I/O) devices via I/O interface 806. The I/O interface 806 may employ communication protocols/methods such as, without limitation, audio, analog, digital, monoaural, RCA, stereo, IEEE-1394, near field communication (NFC), FireWire, Camera Link®, GigE, serial bus, universal serial bus (USB), infrared, PS/2, BNC, coaxial, component, composite, digital visual interface (DVI), high-definition multimedia interface (HDMI), radio frequency (RF) antennas, S-Video, video graphics array (VGA), IEEE 802.n/b/g/n/x, Bluetooth, cellular (e.g., code-division multiple access (CDMA), high-speed packet access (HSPA+), global system for mobile communications (GSM), long-term evolution (LTE), WiMAX, or the like), etc.

Using the I/O interface 806, the computer system 802 may communicate with one or more I/O devices. For example, the input device 808 may be an antenna, keyboard, mouse, joystick, (infrared) remote control, camera, card reader, fax machine, dongle, biometric reader, microphone, touch screen, touchpad, trackball, sensor (e.g., accelerometer, light sensor, GPS, altimeter, gyroscope, proximity sensor, or the like), stylus, scanner, storage device, transceiver, video device/source, visors, etc. Output device 810 may be a printer, fax machine, video display (e.g., cathode ray tube (CRT), liquid crystal display (LCD), light-emitting diode (LED), plasma, or the like), audio speaker, etc. In some embodiments, a transceiver 812 may be disposed in connection with the processor 804. The transceiver may facilitate various types of wireless transmission or reception. For example, the transceiver may include an antenna operatively connected to a transceiver chip (e.g., TEXAS INSTRUMENTS® WILINK WL1286®, BROADCOM® BCM4550IUB8®, INFINEON TECHNOLOGIES® X-GOLD 1436-PMB9800® transceiver, or the like), providing IEEE 802.11a/b/g/n, Bluetooth, FM, global positioning system (GPS), 2G/3G HSDPA/HSUPA communications, etc.

In some embodiments, the processor 804 may be disposed in communication with a communication network 816 via a network interface 814. The network interface 814 may communicate with the communication network 816. The network interface may employ connection protocols including, without limitation, direct connect, Ethernet (e.g., twisted pair 10/100/1000 Base T), transmission control protocol/internet protocol (TCP/IP), token ring, IEEE 802.11a/b/g/n/x, etc. The communication network 816 may include, without limitation, a direct interconnection, local area network (LAN), wide area network (WAN), wireless network (e.g., using Wireless Application Protocol), the Internet, etc. Using the network interface 814 and the communication network 816, the computer system 802 may communicate with devices 818, 820, and 822. These devices may include, without limitation, personal computer(s), server(s), fax machines, printers, scanners, various mobile devices such as cellular telephones, smartphones (e.g., APPLE® IPHONE®, BLACKBERRY® smartphone, ANDROID® based phones, etc.), tablet computers, eBook readers (AMAZON® KINDLER, NOOK® etc.), laptop computers, notebooks, gaming consoles (MICROSOFT® XBOX®, NINTENDO® DS®, SONY® PLAYSTATION®, etc.), or the like. In some embodiments, the computer system 802 may itself embody one or more of these devices.

In some embodiments, the processor 804 may be disposed in communication with one or more memory devices 830 (e.g., RAM 826, ROM 828, etc.) via a storage interface 824. The storage interface may connect to memory devices 830 including, without limitation, memory drives, removable disc drives, etc., employing connection protocols such as serial advanced technology attachment (SATA), integrated drive electronics (IDE), IEEE-1394, universal serial bus (USB), fiber channel, small computer systems interface (SCSI), STD Bus, RS-232, RS-422, RS-485, 12C, SPI, Microwire, 1-Wire, IEEE 1284, Intel® QuickPathInterconnect, InfiniBand, PCIe, etc. The memory drives may further include a drum, magnetic disc drive, magneto-optical drive, optical drive, redundant array of independent discs (RAID), solid-state memory devices, solid-state drives, etc.

The memory devices 830 may store a collection of program or database components, including, without limitation, an operating system 832, user interface application 834, web browser 836, mail server 838, mail client 840, user/application data 842 (e.g., any data variables or data records discussed in this disclosure), etc. The operating system 832 may facilitate resource management and operation of the computer system 802. Examples of operating systems include, without limitation, APPLE® MACINTOSH® OS X, UNIX, Unix-like system distributions (e.g., Berkeley Software Distribution (BSD), FreeBSD, NetBSD, OpenBSD, etc.), Linux distributions (e.g., RED HAT®, UBUNTU®, KUBUNTU®, etc.), IBM® OS/2, MICROSOFT® WINDOWS® (XP®, Vista®/7/8, etc.), APPLE® IOS®, GOOGLE® ANDROID®, BLACKBERRY® OS, or the like. User interface 834 may facilitate display, execution, interaction, manipulation, or operation of program components through textual or graphical facilities. For example, user interfaces may provide computer interaction interface elements on a display system operatively connected to the computer system 802, such as cursors, icons, check boxes, menus, scrollers, windows, widgets, etc. Graphical user interfaces (GUIs) may be employed, including, without limitation, APPLE® MACINTOSH® operating systems' AQUA® platform, IBM® OS/2®, MICROSOFT® WINDOWS® (e.g., AERO®, METRO®, etc.), UNIX X-WINDOWS, web interface libraries (e.g., ACTIVEX®, JAVA®, JAVASCRIPT®, AJAX®, HTML, ADOBE® FLASH®, etc.), or the like.

In some embodiments, the computer system 802 may implement a web browser 836 stored program component. The web browser may be a hypertext viewing application, such as MICROSOFT® INTERNET EXPLORER®, GOOGLE® CHROME®, MOZILLA® FIREFOX®, APPLE® SAFARI®, etc. Secure web browsing may be provided using HTTPS (secure hypertext transport protocol), secure sockets layer (SSL), Transport Layer Security (TLS), etc. Web browsers may utilize facilities such as AJAX®, DHTML, ADOBE® FLASH®, JAVASCRIPT®, JAVA®, application programming interfaces (APIs), etc. In some embodiments, the computer system 802 may implement a mail server 838 stored program component. The mail server may be an Internet mail server such as MICROSOFT® EXCHANGE®, or the like. The mail server may utilize facilities such as ASP, ActiveX, ANSI C++/C #, MICROSOFT .NET® CGI scripts, JAVA®, JAVASCRIPT®, PERL®, PHP®, PYTHON®, WebObjects, etc. The mail server may utilize communication protocols such as internet message access protocol (IMAP), messaging application programming interface (MAPI), MICROSOFT® EXCHANGE®, post office protocol (POP), simple mail transfer protocol (SMTP), or the like. In some embodiments, the computer system 802 may implement a mail client 840 stored program component. The mail client may be a mail viewing application, such as APPLE MAIL®, MICROSOFT ENTOURAGE®, MICROSOFT OUTLOOK® MOZILLA THUNDERBIRD®, etc.

In some embodiments, computer system 802 may store user/application data 842, such as the data, variables, records, etc. (e.g., a set of first case twins, a set of second case twins, a plurality of patient cases, prompt templates and the like) as described in this disclosure. Such databases may be implemented as fault-tolerant, relational, scalable, secure databases such as ORACLE® OR SYBASE®. Alternatively, such databases may be implemented using standardized data structures, such as an array, hash, linked list, struct, structured text file (e.g., XML), table, or as object-oriented databases (e.g., using OBJECTSTORE®, POET®, ZOPE®, etc.). Such databases may be consolidated or distributed, sometimes among the various computer systems discussed above in this disclosure. It is to be understood that the structure and operation of the any computer or database component may be combined, consolidated, or distributed in any working combination.

Various embodiments provide method and system for generating precision diagnosis and precision treatment pathways based on patient case twins. The disclosed method and system may receive a query patient case from a user device. The query patient case may include a set of query medical parameters corresponding to a patient. Further, the disclosed method and system may identify a first set of case twins corresponding to the query patient case using a retrieval model based on a similarity analysis between the set of query medical parameters and a set of patient case medical parameters of each of a plurality of patient cases stored in a database. The first set of case twins may include a set of similar patient cases to the query patient case. The first set of similar patient cases is a subset of a plurality of patient cases stored in the database. Each of the plurality of patient cases may include a set of patient case medical parameters, patient case diagnosis, and patient case treatment pathway. Further, the disclosed method and system may identify a second set of case twins from the first set of case twins using a Generative Artificial Intelligence (GenAI) model. Moreover, the disclosed method and system may determine a precision diagnosis for the query patient case based on the query patient case and the second set of case twins using the GenAI model. Thereafter, the disclosed method and system may generate a precision treatment pathway for the query patient case based on the query patient case, the second set of case twins, and the determined precision diagnosis using the GenAI model.

Thus, the disclosed method and system try to overcome the technical problem of generating precision diagnosis and precision treatment pathways based on patient case twins. The disclosed method and system may increase accuracy and efficiency of a GenAI model (such as LLM and LMM) by grounding the GenAI model with the query patient case to diagnose a disease and subsequently generate treatment pathway more precisely for a user (e.g., a doctor). The disclosed method and system may provide evidence from clinical literature (e.g., PubMed Central) and historical patients' data to justify the generated treatment pathway. The disclosed method and system may contribute to fostering health equity by generating expert-level diagnosis and treatment recommendations. The disclosed method and system may be cost-effective and more accessible for a rural population. The disclosed method and system may be used by a healthcare provider (such as hospitals, insurance companies, online healthcare consulting service providers, clinical research organizations including pharmaceutical organizations, individual clinicians, etc.). By way of an example, when an insurance company can deploy the healthcare assistance device for partnered hospitals to assist in medication preauthorization. A private hospital chain or health department in a specific country may deploy the healthcare assistance device across their associated hospitals to serve as an AI assistant for physicians. An independent service provider may leverage the healthcare assistance device to provide paid services for independent clinicians. The disclosed method and system may deploy through cloud or on premises. By way of an example, when cloud-based deployment is done, then input from the user may be uploaded on the cloud, computation may be done on the cloud, and the responses may also be sent to the user.

In light of the above-mentioned advantages and the technical advancements provided by the disclosed method and system, the claimed steps as discussed above are not routine, conventional, or well understood in the art, as the claimed steps enable the following solutions to the existing problems in conventional technologies. Further, the claimed steps clearly bring an improvement in the functioning of the device itself as the claimed steps provide a technical solution to a technical problem.

It will be appreciated that, for clarity purposes, the above description has described embodiments of the invention with reference to different functional units and processors. However, it will be apparent that any suitable distribution of functionality between different functional units, processors or domains may be used without detracting from the invention. For example, functionality illustrated to be performed by separate processors or controllers may be performed by the same processor or controller. Hence, references to specific functional units are only to be seen as references to suitable means for providing the described functionality, rather than indicative of a strict logical or physical structure or organization.

Although the present invention has been described in connection with some embodiments, it is not intended to be limited to the specific form set forth herein. Rather, the scope of the present invention is limited only by the claims. Additionally, although a feature may appear to be described in connection with particular embodiments, one skilled in the art would recognize that various features of the described embodiments may be combined in accordance with the invention.

Furthermore, one or more computer-readable storage media may be utilized in implementing embodiments consistent with the present disclosure. A computer-readable storage medium refers to any type of physical memory on which information or data readable by a processor may be stored. Thus, a computer-readable storage medium may store instructions for execution by one or more processors, including instructions for causing the processor(s) to perform steps or stages consistent with the embodiments described herein. The term “computer-readable medium” should be understood to include tangible items and exclude carrier waves and transient signals, i.e., be non-transitory. Examples include random access memory (RAM), read-only memory (ROM), volatile memory, nonvolatile memory, hard drives, CD ROMs, DVDs, flash drives, disks, and any other known physical storage media.

It is intended that the disclosure and examples be considered as exemplary only, with a true scope and spirit of disclosed embodiments being indicated by the following claims.

What is claimed is:

1. A computer-implemented method for generating precision diagnosis and precision treatment pathways based on patient case twins, the method comprising:
receiving, by a healthcare assistance device from a user interface (UI), a query patient case corresponding to a patient, wherein the query patient case comprises a set of query medical parameters;
identifying, by the healthcare assistance device, a first set of case twins corresponding to the query patient case using a retrieval model based on a similarity analysis between the set of query medical parameters and a set of patient case medical parameters of each of a plurality of patient cases stored in a database, wherein the first set of case twins comprises a set of similar patient cases to the query patient case, wherein the first set of similar patient cases is a subset of a plurality of patient cases stored in the database, and wherein each of the plurality of patient cases comprises a set of patient case medical parameters, patient case diagnosis, and patient case treatment pathway;
identifying, by the healthcare assistance device, a second set of case twins from the first set of similar patient cases using a Generative Artificial Intelligence (GenAI) model;
determining, by the healthcare assistance device, a precision diagnosis for the query patient case based on the query patient case and the second set of case twins using the GenAI model; and
generating, by the healthcare assistance device, a precision treatment pathway for the query patient case based on the query patient case, the second set of case twins, and the determined precision diagnosis using the GenAI model.

2. The method of claim 1, wherein identifying the first set of case twins comprises:
creating, by the healthcare assistance device, a plurality of query medical parameter embeddings from the set of query medical parameters using an embedding model;
calculating, by the healthcare assistance device, a first similarity score between the query patient case and each of the plurality of patient cases based on a distance function-based similarity analysis between the plurality of query medical parameter embeddings and a corresponding plurality of patient case medical parameter embeddings of each of the plurality of patient cases, wherein the plurality of patient case medical parameter embeddings is pre-stored in the database; and
selecting, by the healthcare assistance device, the first set of case twins from the plurality of patient cases based on the first similarity score.

3. The method of claim 1, wherein the identifying the second set of case twins comprises:
providing, by the healthcare assistance device, a case identification prompt to the GenAI model, wherein the case identification prompt comprises the first set of case twins, the set of query medical parameters, and a set of instructions corresponding to the second set of case twins;
generating, by the healthcare assistance device via the GenAI model, an sorted list of the first set of case twins based on the case identification prompt, wherein the sorted list comprises a patient case identifier (ID) of each of the first set of patient cases and a second similarity score between the query patient case and each of the first set of patient cases, wherein the second similarity score is calculated by the GenAI model, and wherein the first set of case twins in the sorted list is arranged based on the second similarity score;
comparing, by the healthcare assistance device, the second similarity score of each of the first set of case twins with a predefined threshold similarity score; and
truncating, by the healthcare assistance device, the sorted list based on the comparison to obtain the second set of case twins.

4. The method of claim 1, further comprising:
randomly selecting, by the healthcare assistance device, a pair of patient cases from the plurality of patient cases stored in the database;
for each pair of patient cases from the plurality of patient cases,
creating, by the healthcare assistance device, a plurality of patient case medical parameter embeddings corresponding to each patient case of the pair of patient cases through an embedding model;
creating, by the healthcare assistance device, a plurality of patient case diagnosis embeddings corresponding to each patient case of the pair of patient cases through the embedding model;
calculating, by the healthcare assistance device, a case similarity score between the plurality of patient case medical parameter embeddings of each patient case of the pair of patient cases using the similarity analysis; and
calculating, by the healthcare assistance device, a diagnosis similarity score between the plurality of patient case diagnosis embeddings of each patient case of the pair of patient cases using the similarity analysis.

5. The method of claim 4, further comprising:
for each pair of patient cases from the plurality of patient cases, and for each weightage variant of a set of weightage variants,
assigning, by the healthcare assistance device, a weight to each of the case similarity score and the diagnosis similarity score, wherein the weight is predefined for the weightage variant;
determining, by the healthcare assistance device, a weighted similarity score between the pair of patient cases from the case similarity score and the diagnosis similarity score based on the assigned weight, wherein the weighted similarity score is a weighted average of the case similarity score and the diagnosis similarity score based on the assigned weight; and
for each weightage variant of a set of weightage variants, generating, by the healthcare assistance device, a fine-tuning dataset for the retrieval model, wherein the fine-tuning dataset comprises each of the plurality of patient cases and the associated weighted similarity score between each pair of patient cases from the plurality of patient cases for the weightage variant.

6. The method of claim 5, further comprising independently fine-tuning, by the healthcare assistance device, the retrieval model using the fine-tuning dataset for each weightage variant of a set of weightage variants.

7. The method of claim 1, wherein determining the precision diagnosis for the query patient case further comprises:
providing, by the healthcare assistance device, a precision diagnosis determination prompt to the GenAI model, wherein the precision diagnosis determination prompt comprises the set of query medical parameters, the second set of case twins, and a set of diagnosis instructions;
generating, by the healthcare assistance device and via the GenAI model, a response to the precision diagnosis determination prompt, wherein the response comprises the precision diagnosis for the query patient case; and
presenting, by the healthcare assistance device, the precision diagnosis on the UI.

8. The method of claim 1, wherein the generating the precision treatment pathway further comprises:
providing, by the healthcare assistance device, a precision treatment generation prompt to the GenAI model, wherein the precision treatment generation prompt comprises the set of query medical parameters, the second set of case twins, and a set of treatment instructions;
generating, by the healthcare assistance device and via the GenAI model, a response to the precision treatment generation prompt, wherein the response comprises the precision treatment pathway for the query patient case; and
presenting, by the healthcare assistance device, the precision treatment pathway on the UI.

9. The method of claim 1, wherein each of the set of query medical parameters and the set of patient case medical parameters comprises demographic information, medical history, symptoms, physical examination information, and laboratory test results.

10. A system for generating precision diagnosis and precision treatment pathways based on patient case twins, the system comprising:
a processor; and
a memory communicatively coupled to the processor, wherein the memory stores processor executable instructions, which, on execution, causes the processor to:
receive, from a User Interface (UI), a query patient case corresponding to a patient, wherein the query patient case comprises a set of query medical parameters;
identify a first set of case twins corresponding to the query patient case using a retrieval model based on a similarity analysis between the set of query medical parameters and a set of patient case medical parameters of each of a plurality of patient cases stored in a database, wherein the first set of case twins comprises a set of similar patient cases to the query patient case, wherein the first set of similar patient cases is a subset of a plurality of patient cases stored in the database, and wherein each of the plurality of patient cases comprises a set of patient case medical parameters, patient case diagnosis, and patient case treatment pathway;
identify a second set of case twins from the first set of similar patient cases using a GenAI model;
determine a precision diagnosis for the query patient case based on the query patient case and the second set of case twins using the GenAI model; and
generate a precision treatment pathway for the query patient case based on the query patient case, the second set of case twins, and the determined precision diagnosis using the GenAI model.

11. The system of claim 10, wherein identifying the first set of case twins, the processor executable instructions further cause the processor to:
create a plurality of query medical parameter embeddings from the set of query medical parameters using an embedding model;
calculate a first similarity score between the query patient case and each of the plurality of patient cases based on a distance function-based similarity analysis between the plurality of query medical parameter embeddings and a corresponding plurality of patient case medical parameter embeddings of each of the plurality of patient cases, wherein the plurality of patient case medical parameter embeddings is pre-stored in the database; and
select the first set of case twins from the plurality of patient cases based on the first similarity score.

12. The system of claim 10, wherein the identifying the second set of case twins, the processor executable instructions further cause the processor to:
provide a case identification prompt to the GenAI model, wherein the case identification prompt comprises the first set of case twins, the set of query medical parameters, and a set of instructions corresponding to the second set of case twins;
generate, via the GenAI model, an sorted list of the first set of case twins based on the case identification prompt, wherein the sorted list comprises a patient case ID of each of the first set of patient cases and a second similarity score between the query patient case and each of the first set of patient cases, wherein the second similarity score is calculated by the GenAI model, and wherein the first set of case twins in the sorted list is arranged based on the second similarity score;
compare the second similarity score of each of the first set of case twins with a predefined threshold similarity score; and
truncate the sorted list based on the comparison to obtain the second set of case twins.

13. The system of claim 10, wherein the processor executable instructions further cause the processor to:
randomly select a pair of patient cases from the plurality of patient cases stored in the database;
for each pair of patient cases from the plurality of patient cases,
create a plurality of patient case medical parameter embeddings corresponding to each patient case of the pair of patient cases through an embedding model;
create a plurality of patient case diagnosis embeddings corresponding to each patient case of the pair of patient cases through the embedding model;
calculate a case similarity score between the plurality of patient case medical parameter embeddings of each patient case of the pair of patient cases using the similarity analysis; and
calculate a diagnosis similarity score between the plurality of patient case diagnosis embeddings of each patient case of the pair of patient cases using the similarity analysis.

14. The system of claim 13, wherein the processor executable instructions further cause the processor to:
for each pair of patient cases from the plurality of patient cases, and for each weightage variant of a set of weightage variants,
assign a weight to each of the case similarity score and the diagnosis similarity score, wherein the weight is predefined for the weightage variant;
determine a weighted similarity score between the pair of patient cases from the case similarity score and the diagnosis similarity score based on the assigned weight, wherein the weighted similarity score is a weighted average of the case similarity score and the diagnosis similarity score based on the assigned weight; and
for each weightage variant of a set of weightage variants, generate a fine-tuning dataset for the retrieval model, wherein the fine-tuning dataset comprises each of the plurality of patient cases and the associated weighted similarity score between each pair of patient cases from the plurality of patient cases for the weightage variant.

15. The system of claim 14, wherein the processor executable instructions further cause the processor to independently fine-tune the retrieval model using the fine-tuning dataset for each weightage variant of a set of weightage variants.

16. The system of claim 10, wherein determining the precision diagnosis for the query patient case, wherein the processor executable instructions further cause the processor to:
provide a precision diagnosis determination prompt to the GenAI model, wherein the precision diagnosis determination prompt comprises the set of query medical parameters, the second set of case twins, and a set of diagnosis instructions;
generate, via the GenAI model, a response to the precision diagnosis determination prompt, wherein the response comprises the precision diagnosis for the query patient case; and
present the precision diagnosis on the UI.

17. The system of claim 10, wherein the generating the precision treatment pathway, the processor executable instructions further cause the processor to:
provide a precision treatment generation prompt to the GenAI model, wherein the precision treatment generation prompt comprises the set of query medical parameters, the second set of case twins, and a set of treatment instructions;
generate, via the GenAI model, a response to the precision treatment generation prompt, wherein the response comprises the precision treatment pathway for the query patient case; and
present the precision treatment pathway on the UI.

18. The system of claim 10, wherein each of the set of query medical parameters and the set of patient case medical parameters comprises demographic information, medical history, symptoms, physical examination information, and laboratory test results.

19. A non-transitory computer-readable medium storing computer-executable instructions for generating precision diagnosis and precision treatment pathways based on patient case twins, the computer-executable instructions configured for:
receiving, from a UI, a query patient case corresponding to a patient, wherein the query patient case comprises a set of query medical parameters;
identifying a first set of case twins corresponding to the query patient case using a retrieval model based on a similarity analysis between the set of query medical parameters and a set of patient case medical parameters of each of a plurality of patient cases stored in a database, wherein the first set of case twins comprises a set of similar patient cases to the query patient case, wherein the first set of similar patient cases is a subset of a plurality of patient cases stored in the database, and wherein each of the plurality of patient cases comprises a set of patient case medical parameters, patient case diagnosis, and patient case treatment pathway;
identifying a second set of case twins from the first set of similar patient cases using a GenAI model;
determining a precision diagnosis for the query patient case based on the query patient case and the second set of case twins using the GenAI model; and
generating a precision treatment pathway for the query patient case based on the query patient case, the second set of case twins, and the determined precision diagnosis using the GenAI model.

20. The non-transitory computer-readable medium of claim 19, wherein identifying the first set of case twins, the computer-executable instructions are further configured for:
creating a plurality of query medical parameter embeddings from the set of query medical parameters using an embedding model;
calculating a first similarity score between the query patient case and each of the plurality of patient cases based on a distance function-based similarity analysis between the plurality of query medical parameter embeddings and a corresponding plurality of patient case medical parameter embeddings of each of the plurality of patient cases, wherein the plurality of patient case medical parameter embeddings is pre-stored in the database; and
selecting the first set of case twins from the plurality of patient cases based on the first similarity score.
